(19) United States
(12) Patent Application Publication
(10) Pub. No.: US 2023/0317265 A1
(43) Pub. Date: Oct. 5, 2023

(54) MEDICAL SYSTEM AND PROCESS

(71) Applicant: Drägerwerk AG & Co. KGaA, Lübeck (DE)

(72) Inventors: Thomas KRÜGER, Lübeck (DE); Frank FRANZ, Lübeck (DE); Birger LANDWEHR, Lübeck (DE)

(21) Appl. No.: 18/192,750

(22) Filed: Mar. 30, 2023

(30) Foreign Application Priority Data
Apr. 4, 2022 (DE) 10 2022 107 947.3

(51) Int. Cl. G16H 40/40

(52) U.S. Cl. CPC G16H 40/40

(57) ABSTRACT

A medical system (100) provides a setting recommendation (105) for a ventilator. A data interface (110) receives static patient data that includes values of patient parameters that are substantially invariant during a treatment period. A measurement interface (120) receives dynamic patient data that includes values of patient parameters that change during the treatment period. A processing unit (150) determines a new current patient data set based on the currently available patient data immediately after receiving the patient data and initiates, triggered by the determination of the new current patient data set, a comparison between the values of the current patient data set and stored assignment rules from a rule memory. Based on the comparison, a recommended device setting (134) of the ventilator, in view of the current patient data set, is received from a rule memory unit. A recommendation is output based on a comparison with a current device setting.

### CROSS REFERENCE TO RELATED APPLICATIONS

This application claims the benefit of priority under 35 U.S.C. §119 of German Application 10 2022 107 947.3, filed Apr. 4, 2022, the entire contents of which are incorporated herein by reference.

### TECHNICAL FIELD

The invention relates to a medical system for providing a setting recommendation for a ventilator for ventilating a patient. Furthermore, the invention relates to a process for providing a setting recommendation for a ventilator for ventilating a patient.

### BACKGROUND

In principle, the determination of recommendations for ventilator operation is known. For example, automatic recognition of body metrics such as height and weight for subsequent adjustment of alarm settings is known. Also known is the use of so-called expert systems, in which a treatment recommendation is issued based on physiological models and/or a corresponding treatment is carried out automatically.

In the following, a ventilator is understood to be a device which ventilates a patient. So, for example, in addition to devices that are used exclusively for ventilation, anesthesia devices are also ventilation devices within the meaning of this invention.

### SUMMARY

An object of the present invention is to provide an improved medical system, in particular a system that arrives at treatment recommendations in a comprehensible and rapid manner.

According to the invention, a medical system for providing a setting recommendation for a ventilator for ventilating a patient is provided for solving this task, comprising a data interface, a measured value interface, a control memory unit, a processing unit, and an output unit.

The data interface is configured to receive static patient data from input data or stored data, such as from at least one patient database, wherein static patient data comprises values of patient parameters that are substantially invariant over the course of a treatment period.

The measurement interface is configured to receive dynamic patient data from ventilator related measured data, input data or stored ventilator data, such as from at least one ventilator, wherein dynamic patient data includes values of patient parameters that change over the course of treatment.

A plurality of assignment rules (mapping rules) are stored in the rule memory unit, wherein an assignment rule comprises an assignment between at least one range of values for a patient parameter and a recommended device setting for at least one ventilator parameter of the ventilator.

The processing unit is configured to receive the static patient data and the dynamic patient data and to determine therefrom, in each case immediately after the receipt of corresponding patient data, a new current patient data set based on the currently available patient data, the processing unit being further configured to initiate, triggered by the determination of the new current patient data set, a comparison between the values of the current patient data set and the stored assignment rules and, based on this comparison, to receive at least one recommended device setting of the ventilator, recommended in view of the current patient data set from the rule memory unit, and wherein the processing unit is configured to compare the at least one recommended device setting with a current device setting of the ventilator and to initiate a recommendation output based on this comparison, wherein the recommendation output indicates the setting recommendation for the at least one ventilation parameter based on the at least one recommended device setting.

The output unit is configured to receive the recommendation output and graphically output the corresponding setting recommendation via a display.

In the context of the invention, it was recognized that a setting recommendation must be made quickly in everyday clinical practice. Therefore, upon receipt of patient data, the processing unit immediately checks whether a new device setting should be recommended. In particular, dynamic patient data is not first required for an initial recommendation. After the first reception of patient data, such as static patient data like age or weight, the medical system can already output the recommendation according to the stored assignment rules.

The use of known assignment rules, also enables the treatment recommendation to be derived in a comprehensible manner. Thus, the direct association between the range of values of a patient parameter and the corresponding device setting via the assignment rule enables a simple understanding of the operation of the medical system according to the invention. This is advantageous in supporting a user’s confidence in the system.

The components of the medical system according to the invention can be arranged at least partially spatially separated, in particular in different housings. Alternatively, all components of the medical system according to the invention may also be arranged in a common housing. The various components of the medical system according to the invention are separated from one another at least at the software level.

Devices for providing patient data are known to the person skilled in the art in this field, so that, in addition to some examples mentioned below, further patient data and devices for providing such patient data can also be used by the medical system. In this context, the determination of which patient data is static patient data and which is dynamic patient data is made prior to a provision of the medical system according to the invention. For example, weight belongs to static patient data, although a very large fluctuation in weight can be possible for newborns. This example shows that receiving static but possibly variable patient data on a regular basis may also be useful. The measurement interface and data interface may be configured to be the same. In particular, the two interfaces may be configured as a common interface, differing from each other only at the software level. Both interfaces enable the data to be received and further processed by the processing unit according to the invention.

The comparison between the recommended device setting and the current device setting can be, for example, the comparison of a recommended ventilation parameter with a currently present ventilation parameter. For example, in one embodiment, the recommendation output may be triggered only if the recommended ventilation parameter differs from the currently present ventilation parameter by more than 5%, in particular by more than 10%. In a further embodiment, a difference between a recommended ventilation mode and a currently present ventilation mode is detected in this comparison and a corresponding recommendation output is triggered.

Preferred embodiments of the medical system according to the invention are described below.

Preferably, the medical system according to the invention further comprises a user interface configured to receive an applicable assignment rule via a user input. Receiving the assignment rule to be applied via the user interface may in particular comprise modifying a displayed already existing assignment rule. The user input can take place on a different device than the reception of the patient data.

In a particularly preferred embodiment of the medical system, the comparison between the at least one recommended device setting and the current device setting of the ventilator only triggers the recommendation output if a recommended setting value for the at least one ventilation parameter and a current setting value of the ventilation parameter to be set have a predetermined minimum setting value difference from one another. This advantageously ensures that a recommendation output is not displayed for very small setting value differences, which could then distract the user from other important information of the ventilator. It can be assumed that the user is preferably only interested in the recommendation output if the recommended change of the device setting could cause a serious improvement of the treatment. Therefore, the provision of the predetermined setting value minimum difference (threshold) in this embodiment is particularly advantageous to keep the number of recommendation outputs low and, consequently, to allow serious user engagement with the recommendation output. The minimum setpoint difference can be a predetermined absolute value and/or a relative deviation between the recommended setpoint value and the existing setpoint value.

In a particularly preferred embodiment, the recommendation output additionally indicates the assignment rule used for the at least one recommended device setting, with the output unit being further configured to output the assignment rule used at least partially graphically via the display. In this way, the user can particularly advantageously recognize the reason for the recommendation output and thereby decide whether the user wants to make a change to the device setting based on the assignment rule used. This allows the user to critically recognize and evaluate the reason for the recommendation output. For example, the user may reject the recommendation output based on individually present reasons, such as: a current change in the patient’s condition, such as after waking up; a personal rejection of the stored assignment rule; an effect outweighing the benefits of the recommended device setting if the current device setting is retained. Another advantage of the present embodiment is the supported confidence in the recommendation output, since the user can transparently see why this recommendation output occurred. This allows a particularly good evaluation of whether the recommendation output should be followed or not. In addition, the output in this embodiment allows a quick recognition of a changed state of the treatment, which may have led to the recommendation output.

In an advantageous variant of the preceding embodiment, the processing unit is further configured to output at least two recommended device settings with the corresponding assignment rule used via the recommendation output if, for these at least two recommended device settings, the corresponding recommended setting value and the setting value currently present have the respective predetermined setting value minimum difference from one another, and the output unit being configured to output the assignment rules used for the at least two recommended device settings at least partially graphically via the display in addition to the setting recommendation. In this variant, it is ensured that the user themself can decide between two recommended device settings or keeping the current setting. This variant clarifies that the medical system according to the invention does not make a decision for the user, but outputs various recommendations as well as their reasons in the form of the assignment rules in an unfiltered manner. Thus, the user can decide for themself how the treatment should proceed further and/or which applied assignment rule has the greater relevance for the current treatment.

In a preferred embodiment, at the start of ventilation by the ventilator, the processing unit is configured to determine the current patient data set immediately after the first received value of a patient parameter and to trigger the recommendation output depending on the comparison between the recommended device setting and the current device setting. In this embodiment, an initial recommendation output is provided particularly quickly, so that the medical system could help by providing a recommendation output already at the beginning of a treatment.

In an advantageous variant of the preceding embodiment, when ventilation is started by the ventilator, the processing unit is configured to take into account static patient data received in accordance with a predetermined processing hierarchy in time before dynamic patient data received for determining the current patient data set. Static patient data can be evaluated particularly easily and immediately. Therefore, this variant allows a particularly fast provision of an initial setting recommendation, which is then possibly changed again somewhat later by taking the dynamic patient data into account. This ensures that the recommendation output is not delayed until a large amount of patient data has been determined and evaluated. It also ensures that even if components of the ventilation system in use have not yet been activated, such as a sensor that has not yet been properly attached, a setting recommendation based on static patient data can be received immediately to ensure a sensible and efficient start to treatment.

Preferably, the static patient data pertains to a group of patient parameters that includes at least one of the following: patient category; height; age; gender; weight; ventilation mode; pre-existing condition. Such patient data are typically recorded at the beginning of a treatment and change little during the course of a treatment.

Preferably, the dynamic patient data pertains to a group of patient parameters that includes at least one of the following: ventilation sensor data; diagnostic data; blood gas values; medication; hemodynamic index; bed position; current ventilation maneuver; data derived from sensor data, such as tidal volume and/or minute volume. Such patient data is typically recorded repeatedly, particularly substantially continuously recorded. In the context of the present invention, such rapidly changing patient data provides for the output of a setting recommendation during treatment, whereas static patient data typically has great relevance to the output of the setting recommendation at the beginning of a treatment.

In a particularly preferred embodiment, the stored assignment rules are in the form of an “if-then” case distinction. Such a form of the assignment rules allows a particularly simple recording of the reasons for the recommendation output, in particular if the applied assignment rule is also output. Such simply structured assignment rules allow easy input and/or correction of the assignment rules via the user interface. In addition, such a simple structure of the assignment rules reduces the risk of an incorrectly stored assignment rule.

In a further preferred embodiment, the processing of the patient data by the medical system is triggered by a receipt of a trigger signal. Such a trigger signal may, for example, indicate an activation of the medical system according to the invention. Alternatively, or additionally, the start of treatment, for example by the receipt of initial patient data, may trigger the trigger signal and thereby activate the processing of the patient data to provide a setting recommendation. This can therefore ensure that the recommendation outputs are issued automatically from a predetermined point in time that can be tracked by the user.

In a particularly advantageous embodiment, the user interface is further configured to receive an acceptance of a recommended device setting triggered by a user of the medical system and to output a corresponding acceptance signal to the ventilator. In this embodiment, the current device setting of the ventilator can be changed in accordance with the recommended setting in a particularly simple and user-friendly manner. Especially in case of a recommended adjustment of several setting values, the treatment of the patient can thus be configured to the recommended treatment by a single user interaction.

Preferably, the medical system according to the invention further comprises the ventilator, wherein the ventilator outputs present ventilator data to a user of the medical system via the output unit. Consequently, the output unit according to the invention is the same unit that outputs the usual ventilator data. Therefore, this embodiment advantageously allows the avoidance of multiple displays and consequently a simple usability of the medical system. In the context of the present invention, a ventilator means a medical device for ventilating a patient. For example, in addition to a conventional ventilator, an anesthesia machine is also a ventilator within the meaning of the present invention and consequently may also be a component of the medical system according to the present invention.

Preferably, the processing unit is configured to output a data request to a connected medical device after a predetermined waiting time during which no patient data has been received. In this embodiment, it is avoided that no recommendation output according to the invention is enabled due to an undetected error, such as a connection problem between two devices. For example, the data request may result in a request to the user of the system to re-enter or enter for the first time certain static patient data and/or to verify the connection to a particular device, such as a sensor for capturing dynamic patient data. Therefore, according to this embodiment, the medical system reduces a likelihood of undetected malfunction of the system and/or due to user misoperation.

According to a further aspect of the invention, a process for providing a setting recommendation for a ventilator for ventilating a patient is proposed for solving the above task. The process according to the invention comprises the following steps:
storing a plurality of assignment rules, wherein an assignment rule comprises an assignment between at least one range of values for a patient parameter and a recommended device setting for at least one ventilator parameter of the ventilator;
receiving static patient data, wherein static patient data comprises values of patient parameters that are substantially invariant over the course of a treatment period;
receiving dynamic patient data, wherein dynamic patient data comprises values of patient parameters that change over the course of treatment;
determining a new current patient data set based on the currently available patient data immediately after receiving corresponding patient data;
initiating a comparison between the values of the current patient dataset and the stored assignment rules, triggered by the determination of the new current patient data set;
receiving a recommended ventilator device setting given the current patient data set;
comparing the at least one recommended device setting to a current device setting of the ventilator and triggering a recommendation output based on the comparison, wherein the recommendation output indicates the recommended setting for the at least one ventilation parameter based on the at least one recommended device setting;
receiving the recommendation output and graphically outputting the corresponding setting recommendation.

This process is carried out by the medical system according to the invention and therefore has all the advantages of this system. In particular, it allows a reliable, comprehensible and fast output of the setting recommendation.

The steps of the process according to the invention can be executed at least partially on a common processor or on spatially separated components.

Preferably, according to a predetermined processing hierarchy for the determination of the current patient data set, received static patient data is considered before received dynamic patient data. This enables a particularly fast output of the setting recommendation based on the static patient data, which changes very little, before this setting recommendation is possibly corrected at a later time based on the dynamic patient data.

The invention will now be explained in more detail with reference to advantageous examples of embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of a first embodiment of a medical system according to the invention;

FIG. 2 is a schematic representation of a second embodiment of the medical system according to the invention;

FIG. 3 is a schematic representation of a third embodiment of the medical system according to the invention;

FIG. 4 is a flowchart of an embodiment of a process according to a further aspect of the invention.

### DESCRIPTION OF PREFERRED EMBODIMENTS

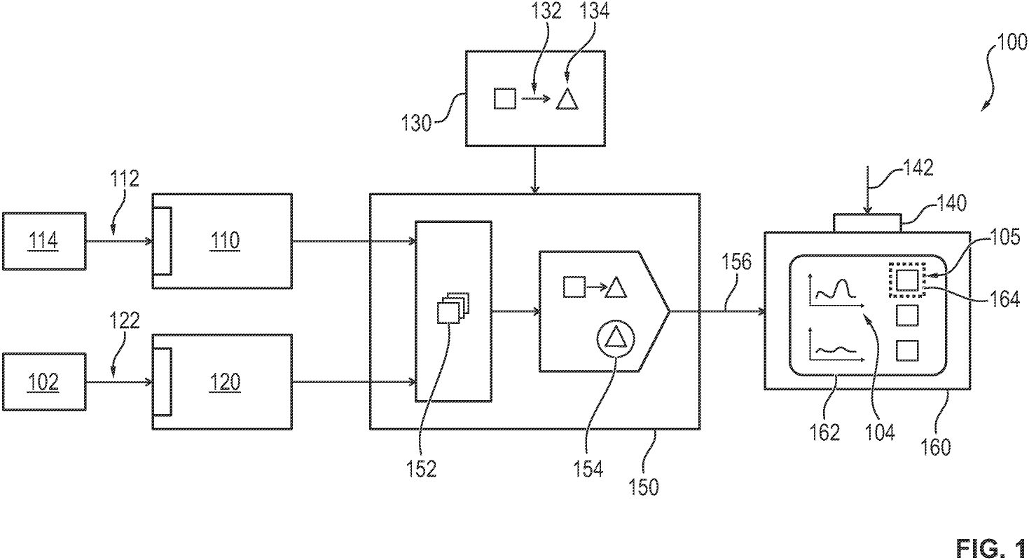

Referring to the drawings, FIG. 1 shows a schematic diagram of a first embodiment of a medical system 100 according to the invention.

The medical system 100 is configured to provide a setting recommendation 105 for a ventilator 102 for ventilating a patient. For this purpose, the medical system 100 comprises at least a data interface 110, a measured value interface 120, a control memory unit 130, a processing unit 150, and an output unit 160. Additionally, in the illustrated embodiment, the medical system 100 comprises a user interface 140. These components of the medical system 100 may be spaced apart from each other or at least partially arranged in a common housing. The subdivision between the various components is thereby at least at the software level.

The data interface 110 is configured to receive static patient data 112. This may be input data such as from at least one patient database 114. The static patient data 112 comprises values of patient parameters that are substantially invariant over the course of a treatment period.

The measured value interface 120 is configured to receive dynamic patient data 122, such as from at least from a ventilator 102. The dynamic patient data 122 comprises values of patient parameters that change over the course of the treatment period. In the present embodiment, the two interfaces 110, 120 are configured as wireless interfaces, such as WLAN, Bluetooth, BLE, ZigBee interface or the like. Alternatively, or additionally, at least one of the two interfaces may be configured as a wired interface. Alternatively, the two interfaces may be configured as a common interface that functions as a measurement interface during the reception of static patient data and as a data interface during the reception of dynamic patient data. The configuration of such interfaces is generally known to the person skilled in the art, so that no further details will be given below.

A plurality of assignment rules (mapping rules) 132 are stored in the rule memory unit 130. A respective assignment rule 132 comprises an assignment between at least one range of values for a patient parameter and a recommended device setting 134 for at least one ventilation parameter of the ventilator 102. The rule memory unit 130 is at least indirectly connected to the user interface 140, which is not shown in FIG. 1 for clarity.

The user interface 140 is configured to receive an applicable assignment rule 132 via a user input 142. The assignment rule to be applied may include, for example, an adjusted originally stored assignment rule, a newly stored assignment rule, or a selection from a group of possible stored assignment rules. In the illustrated embodiment, the user interface 140 is arranged at the output unit 160. Here, the display 162 of the output unit 160 is a touch display which, in combination with possible further buttons, forms the user interface 140 to receive the corresponding user input 142. In an alternative or additional embodiment of the medical system according to the invention, no such user interface for inputting an assignment rule is provided, since, for example, all assignment rules are modified, adapted and/or provided during manufacturing of the system or during maintenance work on the system.

The processing unit 150 is configured to receive the static patient data 112 and the dynamic patient data 122 and to determine therefrom a new current patient data set 152 based on the currently available patient data 112, 122, in each case immediately after receiving corresponding patient data. In this regard, the processing unit 150 is further configured to initiate, triggered by the determination of the new current patient data set 152, a comparison between the values of the current patient data set 152 and the stored assignment rules 132 and, based on this comparison, to receive at least one recommended device setting 134 of the ventilator 132 from the rule memory unit 130 in view of the current patient data set 152. In addition, the processing unit 150 performs a comparison comparing the at least one recommended device setting 134 to a current device setting 154 of the ventilator 102. Based on this comparison, a recommendation output 156 is triggered, wherein the recommendation output 156 indicates the setting recommendation 105 for the at least one ventilator parameter based on the at least one recommended device setting.

In the present case, the output unit 160 is a touch display that graphically outputs the received recommendation output 156 by highlighting a selection area 164 on the touch display. The illustrated highlighting by an outline is merely exemplary. In other embodiments, a separate selection window on the user interface of the ventilator is used as the recommendation output.

A ventilator in the sense of the invention is a device for ventilating a patient, such as an anesthesia machine. For an anesthesia machine, the medical system according to the invention can therefore also be used to output a setting recommendation.

According to the invention, the setting recommendation can indicate an adjustment of a number of setting values of ventilation parameters and/or a change or activation of a specific ventilation mode.

In the illustrated embodiment, the ventilator data 104 is also output from the display of the output unit 160. Through this, a user does not have to check multiple displays to become aware of the outputs of the medical system according to the invention.

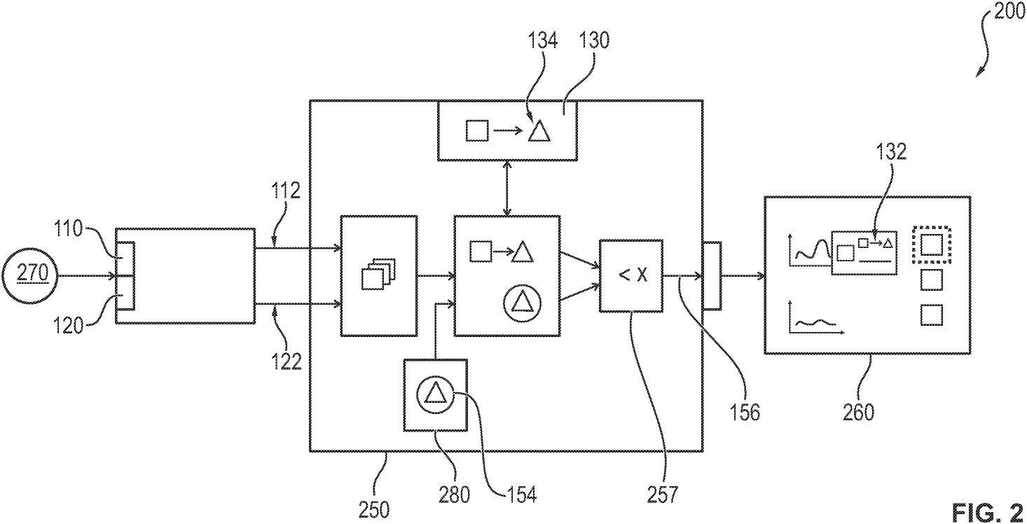

FIG. 2 shows a schematic diagram of a second embodiment of the medical system 200 according to the invention.

The medical system 200 differs from the system 100 shown in FIG. 1 in, among other things, that the data interface 110 and the measurement interface 120 are arranged in a common housing and are separated from each other only at the software level. Thus, the interfaces both receive corresponding patient data via a wireless connection to a network 270, particularly a hospital network. Alternatively, or additionally, the connection to the network may be wired. The two signals shown for transmitting the static patient data 112 and the dynamic patient data 122 may also be provided to the processing unit 250 via a combined signal.

The processing unit 250 is directly connected to the control memory unit 130. In addition, the processing unit has an internal memory 280 on which the currently present device setting 154 is always stored for comparison with the recommended device setting 134.

This comparison is performed in such a way that a recommendation output 156 is only output if a recommended control value for the at least one ventilation parameter and a currently available control value of the ventilation parameter to be set have a predetermined minimum control value difference 257 from one another. This predetermined minimum control value difference 257 is at least 2% of the currently available control value, in particular at least 4% of the currently available control value, particularly preferably 6% of the currently available control value.

Furthermore, the output unit 260 differs from the output unit 160 of FIG. 1 in that the assignment rule 132 used for the recommendation output 156 is output graphically via the display of the output unit 260. This allows the user to quickly see why the displayed recommended device setting 134 has been presented. Particularly advantageously, the illustrated embodiment example of the medical system 200 is configured to output the assignment rule on which the recommendation is based for both, when outputting two different recommended device settings. In this way, one setting recommendation is not filtered out in a non-transparent manner between two equally valid setting recommendations without intervention of the user, but a selection is made possible by the informed user. This underlines an essential aspect of the present invention, which is that the medical system does not make automated decisions but only issues recommendations to the user, which in this example are advantageously supported by the background of the recommendation, namely the assignment rule.

Advantageously, the illustrated medical system 200 is configured to perform the comparison between recommended device setting and current device setting with the receipt of the first patient data at the beginning of a treatment and, if necessary, to output the recommendation output 156. For this purpose, the processing unit 250 has a processing hierarchy of the patient data, according to which received inpatient patient data are taken into account in time before received dynamic patient data for determining the current patient data set. Thus, advantageously, a recommendation output based on inpatient patient data can be provided very quickly, which can directly draw attention to an initially incorrect setting of the ventilator. This avoids the need to collect a large amount of data before a recommendation can be made by the system.

Static patient data for purposes of the present invention may include, for example: patient category; height; age; gender; weight; ventilation mode; pre-existing conditions.

Dynamic patient data as defined by the present invention may include, for example: ventilation sensor data; diagnostic data; blood gas values; medication; hemodynamic index; bed position; current ventilation maneuver; data derived from sensor data such as tidal volume and/or minute volume.

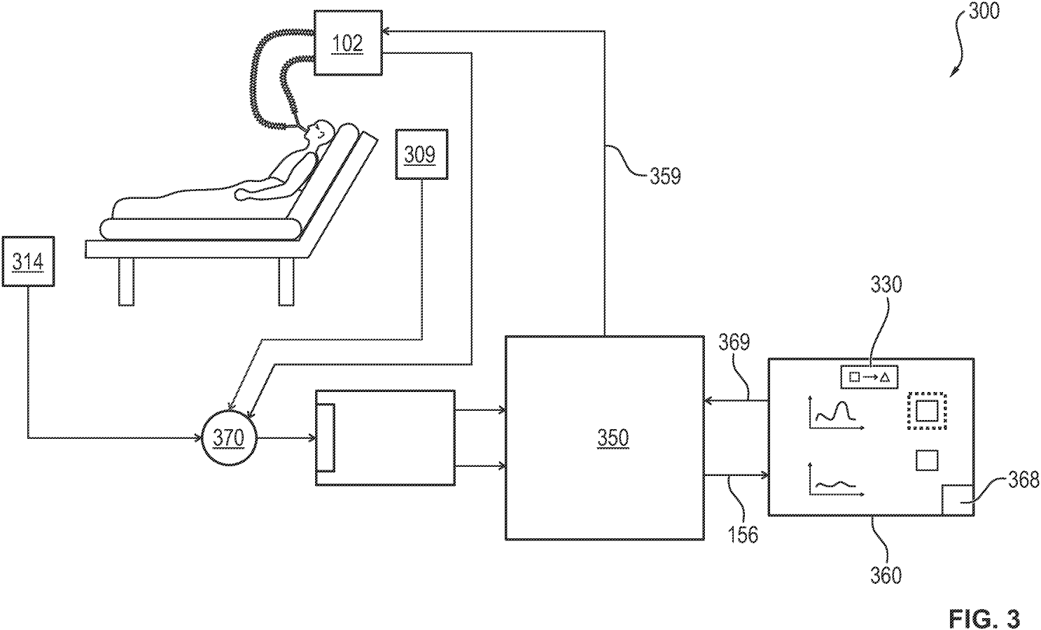

FIG. 3 shows a schematic diagram of a third embodiment of the medical system 300 according to the invention.

The medical system 300 shown differs from the medical system 200 shown in FIG. 2 in that a trigger area 368 is shown on the display of the output unit 360, through which the processing of the patient data according to the invention can be triggered when touched. This allows the user to advantageously decide themself whether a recommendation output 156 is issued or not.

As already shown in FIG. 2, the recommendation output 156 is output as an output window with the assignment rule currently leading to the recommendation. The assignment rules from the rule memory unit, which is not shown, are “if-then” case distinctions in the present case. For example, an assignment rule may be that if a measured value of a patient parameter exceeds a predetermined threshold, a predetermined ventilation mode should be activated. Accordingly, if this mode is detected as not previously activated by the processing unit 350, this would result in a recommendation output 156 that this ventilation mode should be activated given the corresponding assignment rule. Alternatively, or additionally, the assignment rules used may result from so-called look-up tables, which are known, for example, as internal guidelines for certain treatments.

Furthermore, the user interface 330 arranged at the output unit 360 is configured to receive, via a corresponding input area, an acceptance of a recommended device setting triggered by a user of the medical system and to output a corresponding acceptance signal 369 to the ventilator. This output is shown herein only as an acceptance signal to the processing unit 350. The path of the acceptance signal 369 from the processing unit onward to the ventilator is not shown for clarity. The acceptance signal 369 may be routed from the processing unit 350 directly or via another device to the ventilator. In an embodiment not shown, the acceptance signal is output directly to the ventilator from the user interface or output unit.

Finally, the processing unit 350 is also configured to issue a data request 359 to a connected medical device after a predetermined waiting time during which no patient data has been received. In the present case, the data request is made via the network 370, in particular the hospital network. Which device receives the corresponding data request 359 depends on the patient parameter from which no data was received within the waiting time. If, for example, within a predetermined waiting time of at least 10 min, in particular at least 20 min after the start of the treatment of a patient, no data concerning his or her age has been received, the data request 359 is sent to the patient database 314. On the other hand, if, for example, no gas flow data has been received within a predetermined waiting period of at least 20 seconds, in particular at least 1 minute after the last reception of this data, the data request 359 is sent to the sensor unit 309 or the ventilator 102.

In the illustrated embodiment, all near-patient devices are connected to the network 370 and communicate with the medical system 300 according to the invention via this network. Alternatively or additionally, devices may also be at least partially directly connected to the medical system.

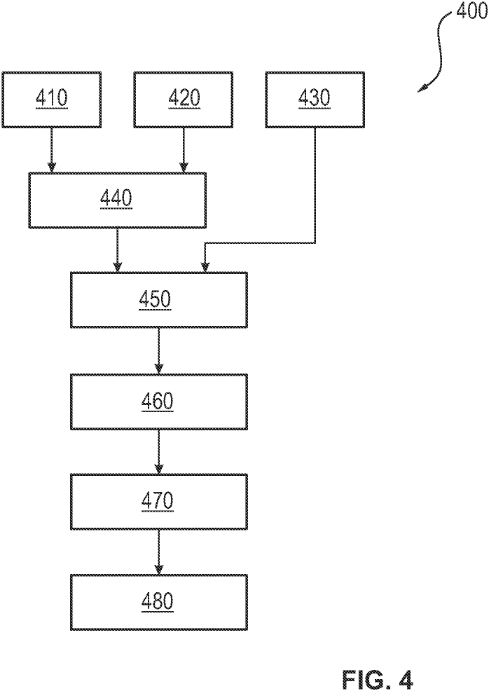

FIG. 4 shows a flowchart of an embodiment of a process 400 according to another aspect of the invention.

The process 400 according to the invention is configured for providing a setting recommendation for a ventilator for ventilating a patient. For this purpose, it has the following process steps.

A first step 410 comprises storing a plurality of assignment rules, wherein an assignment rule comprises an assignment between at least one range of values for a patient parameter and a recommended device setting for at least one ventilator parameter of the ventilator.

A further step 420 comprises receiving static patient data, wherein static patient data comprises values of patient parameters that are substantially invariant over the course of a treatment period.

Another step 430 includes receiving dynamic patient data, wherein dynamic patient data includes values of patient parameters that change over the course of treatment.

A subsequent step 440 includes determining a new current patient data set based on the currently available patient data immediately after receiving corresponding patient data.

A next step 450 includes initiating a comparison between the values of the current patient data set and the stored assignment rules, triggered by determining the new current patient data set.

Another step 460 includes receiving a recommended ventilator device setting given the current patient data set.

A subsequent step 470 includes comparing the at least one recommended device setting to a current device setting of the ventilator and triggering a recommendation output based on the comparison, wherein the recommendation output indicates the recommended setting for the at least one ventilation parameter based on the at least one recommended device setting.

A final step 480 includes receiving the recommendation output and graphically outputting the corresponding setting recommendation.

Steps 410, 420 and 430 can be performed independently of each other. In particular, the memory of the assignment rules according to step 410 can be carried out chronologically before the further steps, for example before the use of the medical system. The further process steps are at least partially based on one another and are therefore preferably carried out in the sequence shown.

In a particularly preferred embodiment, static patient data received is taken into account before dynamic patient data received in accordance with a predetermined processing hierarchy for determining the current patient data set. In this way, a setting recommendation according to the invention can be provided particularly quickly. Preferably, the output of the setting recommendation takes place in less than 20 seconds, in particular in less than 10 seconds, especially preferably in less than 5 seconds after the receipt of the first static patient data.

While specific embodiments of the invention have been shown and described in detail to illustrate the application of the principles of the invention, it will be understood that the invention may be embodied otherwise without departing from such principles.

List of reference characters

| | |
|---|---|
| 100, 200, 300 | Medical system |
| 102 | Ventilator |
| 104 | Ventilator data |
| 105 | Setting recommendation |
| 110 | Data interface |
| 112 | Static patient data |
| 114, 314 | Patient database |
| 120 | Measured value interface |
| 122 | Dynamic patient data |
| 130, 330 | Control memory unit |
| 132 | Assignment rule |
| 134 | Recommended device setting |
| 140 | User interface |
| 142 | User input |
| 150, 250, 350 | Processing unit |
| 152 | Current patient data set |
| 154 | Current device setting |
| 156 | Recommendation output |
| 160, 260, 360 | Output unit |
| 162 | Display |
| 164 | Highlighted selection area |
| 257 | Predetermined minimum setpoint difference |
| 270, 370 | Network |
| 280 | Internal memory |
| 309 | Sensor unit |
| 359 | Data request |
| 368 | Trigger range |
| 369 | Acceptance signal |
| 400 | Procedure |
| 410, 420, 430, 440, 450 | Process steps |
| 460, 470, 480 | |

What is claimed is:

1. A medical system for providing a setting recommendation for a ventilator for ventilating a patient, the medical system comprising:
a data interface configured to receive static patient data, wherein the static patient data comprises values of patient parameters that are substantially invariant over a treatment period;
a measurement interface configured to receive dynamic patient data, wherein the dynamic patient data comprises values of patient parameters that change over the course of treatment;
a rule memory unit in which a plurality of assignment rules are stored, wherein an assignment rule comprises an assignment between at least one range of values for a patient parameter and a recommended device setting for at least one ventilation parameter of the ventilator;
a processing unit which is configured to receive the static patient data and the dynamic patient data and to determine therefrom a new current patient data set based on the currently available patient data in each case immediately after receipt of corresponding patient data, and configured to, triggered by the determination of the new current patient data set, initiate a comparison between values of the new current patient data and the stored assignment rules and, based on the comparison, to receive at least one recommended device setting of the ventilator, which is recommended based on the new patient data set from the rule storage unit, and configured to compare the at least one recommended device setting to a current device setting of the ventilator and to initiate a recommendation output based on the device setting comparison, wherein the recommendation output indicates the setting recommendation for the at least one ventilator parameter based on the at least one recommended device setting; and
an output unit configured to receive the recommendation output and graphically output the corresponding setting recommendation via a display.

2. A medical system according to claim 1, wherein the comparison between the at least one recommended device setting and the current device setting of the ventilator leads to triggering of the recommendation output only if a recommended setting value for the at least one ventilation parameter and a currently present setting value of the ventilation parameter to be set have a predetermined setting value minimum difference from each other.

3. A medical system according to claim 1, wherein:
the recommendation output additionally indicates the assignment rule referenced for the at least one recommended device setting; and
the output unit is further configured to at least partially graphically output the referenced assignment rule via the display.

4. A medical system according to claim 2, wherein:
the processing unit is configured to output at least two recommended device settings, with the corresponding assignment rule used, via the recommendation output if, for the at least two recommended device settings, the corresponding recommended setting value and the currently present setting value have the respective predetermined setting value minimum difference from one another; and
the output unit is configured to output, for the at least two recommended device settings, at least partially graphically via the display, in addition to the setting recommendation, also the assignment rule used.

5. A medical system according to claim 1, wherein the processing unit is configured, upon initiation of a ventilation by the ventilator, to determine the current patient data set immediately after the first received value of a patient parameter and to trigger the recommendation output depending on the comparison between recommended device setting and current device setting.

6. A medical system according to claim 5, wherein upon initiation of ventilation by the ventilator, the processing unit is configured to consider received static patient data in time before received dynamic patient data for determining the current patient data set according to a predetermined processing hierarchy.

7. A medical system according to claim 1, wherein the static patient data relates to a group of patient parameters comprising at least one of the following: patient category; patient height; patient age; patient gender; patient weight; ventilation mode; patient pre-existing condition.

8. A medical system according to claim 1, wherein the dynamic patient data concerns a group of patient parameters comprising at least one of the following parameters: sensor data of ventilation; diagnostic data; blood gas values; medication; hemodynamic index; bed position; current ventilation maneuver; data derived from sensor data, tidal volume and minute volume.

9. A medical system according to claim 1, wherein the stored assignment rules are in the form of an if-then case discrimination.

10. A medical system according to claim 1, wherein processing of the patient data by the medical system is triggered by a receipt of a trigger signal.

11. A medical system according to claim 1, further comprising a user interface configured to receive, via a user input, an assignment rule to be applied.

12. A medical system according to claim 11, wherein the user interface is further configured to receive an acceptance of a recommended device setting triggered by a user of the medical system and to output a corresponding acceptance signal to the ventilator.

13. A medical system according to claim 1, further comprising the ventilator, wherein the ventilator outputs present ventilator data to a user of the medical system via the output unit.

14. A medical system according to claim 1, wherein the processing unit is configured to issue a data request to a connected medical device after a predetermined waiting period in which no patient data has been received.

15. A process for providing a setting recommendation for a ventilator for ventilating a patient, the process comprising the steps of:
storing a plurality of assignment rules, wherein an assignment rule comprises an assignment between at least one range of values for a patient parameter and a recommended device setting for at least one ventilator parameter of the ventilator;
receiving static patient data, wherein static patient data comprises values of patient parameters that are substantially invariant over the course of a treatment period;
receiving dynamic patient data, wherein dynamic patient data includes values of patient parameters that change over the course of treatment;
determine a new current patient data set based on currently available patient data immediately after receiving corresponding patient data;
initiating a comparison between the values of the current patient data set and the stored assignment rules, triggered by the determination of the new current patient data set;
receiving a recommended ventilator device setting given the current patient data set;
comparing the at least one recommended device setting to a current device setting of the ventilator and triggering a recommendation output based on the device setting comparison, wherein the recommendation output indicates the recommended setting for the at least one ventilation parameter based on the at least one recommended device setting; and
receiving the recommendation output and graphically outputting the corresponding setting recommendation.

16. A process according to claim 15, wherein according to a predetermined processing hierarchy for determining the current patient data set, received static patient data is considered temporally before received dynamic patient data is considered.
